(19) United States
(12) Patent Application Publication
(10) Pub. No.: US 2024/0358890 A1
(43) Pub. Date: Oct. 31, 2024

(54) MINERALIZED COLLAGEN MATERIAL, PREPARATION METHOD THEREFOR, AND APPLICATION THEREOF

(71) Applicant: SHANGHAI TENTH PEOPLE’S HOSPITAL, Shanghai (CN)

(72) Inventors: Feng Chen, Shanghai (CN); Yunfei Zhao, Shanghai (CN); Bingqiang Lu, Shanghai (CN); Xinyu Zhao, Shanghai (CN); Shisheng He, Shanghai (CN)

(73) Assignee: SHANGHAI TENTH PEOPLE’S HOSPITAL, Shanghai (CN)

(21) Appl. No.: 18/686,600

(22) PCT Filed: Jan. 1, 2023
(86) PCT No.: PCT/CN2023/071761

§ 371 Date: Feb. 26, 2024

(30) Foreign Application Priority Data
Mar. 29, 2022 (CN) 202210319532.6

(51) Int. Cl. A61L 27/24; A61L 27/02; A61L 27/12; A61L 27/52; A61L 27/54; A61L 27/56; C07K 14/78

(52) U.S. Cl. CPC A61L 27/24; A61L 27/025; A61L 27/12; A61L 27/52; A61L 27/54; A61L 27/56; C07K 14/78; A61L 2300/112; A61L 2300/252; A61L 2300/412; A61L 2430/02

(57) ABSTRACT

The present invention relates to a mineralized collagen material, a preparation method therefor, and an application thereof. The preparation method comprises: dissolving type I collagen, a calcium source, and acid in a first solvent to obtain a first solution; dissolving alkali and a phosphorus source in a second solvent to obtain a second solution; under the condition that a temperature is less than or equal to 45° C., adding the second solution into the first solution and mixing to obtain a third solution; and placing the third solution in a deionized water solvent or an ethanol solvent for soaking, and removing impurities to obtain a mineralized collagen gel. The method has the advantages that: by introducing glycerol into a reaction system, the assembly speed and an assembly structure of the collagen can be controlled, and the nucleation and crystallization speed of a mineral substance and a structure thereof can be controlled, so that the collagen can be assembled into a large-size space network structure at a controllable speed, and mineralization is deeply performed at the same time, so as to prepare continuous large-size mineralized collagen blocks; and high mineralization of the collagen is implemented in a very small reaction system, and the collagen gel can be prepared without repeated replacement of a mineralizing solution, a macromolecular additive, and extra cross-linking.

### TECHNICAL FIELD

The present invention relates to the technical field of material synthesis, and in particular to a mineralized collagen material, preparation method and use thereof.

### BACKGROUND

A considerable part of bone defects caused by trauma, infection, tumors, etc. cannot be repaired by themselves. Bone transplantation and bioengineering materials are the main means to solve this problem. Although bone transplantation, especially autologous bone transplantation, is effective and is a gold standard for the treatment of bone defects, it has the following deficiencies: insufficient supply of autologous bone, various complications related to bone removal surgery, allograft bone rejection, disease transmission, etc., which limits the further popularization of bone transplantation technology.

By comparison, bioengineering materials can overcome the deficiencies of bone transplantation technology. Among the many bioengineering materials, mineralized collagen has become one of the most interesting bioengineering materials in the field of bone regeneration due to its good biocompatibility, bioactivity, bone-promoting ability, and similarity to human bone tissue in terms of composition and structure.

The current collagen mineralization process comprises two steps: collagen molecular assembly and collagen mineralization. Therefore, the methods can be roughly divided into two types: (1) the collagen assembly structure is produced firstly and then the mineralization is performed: (2) the structure is assembled and mineralized simultaneously. At present, both types of methods have made some progress, but there are still many problems, for example, it is difficult to form a self-supporting large-scale three-dimensional structure, that is, the products are in powdery, granular or loose aggregation: the uniformity of mineralization is not high, that is, although some collagen fibers in the product are mineralized, a large part of the collagen fibers is still exposed; and, most methods with better mineralization effects have the defects of complicated steps, low efficiency, difficulty in large-scale production and high industrialization costs. Therefore, there still needs some significant improvement in the preparation of mineralized collagen.

At present, no effective solutions have been proposed for the problems existing in related technologies such as poor mineralization uniformity, complicated preparation steps, low efficiency, inability to produce large-scale products, and high industrialization costs.

### SUMMARY

In view of the defects of the prior art, the present application provides a mineralized collagen material, preparation method and use thereof, so as to at least solve the problems of poor mineralization uniformity, complicated preparation steps, low efficiency, inability to produce large-scale products, and high industrialization costs.

A first aspect of the present invention provides a method for preparing a mineralized collagen material, comprising the steps of:
dissolving type I collagen, calcium source and acid in a first solvent to obtain a first solution, in which the first solvent is glycerin or a mixed solvent of glycerin and water;
dissolving alkali and phosphorus source in a second solvent to obtain a second solution, in which the second solvent is glycerin, water or a mixed solvent of glycerin and water;
adding the second solution to the first solution at a temperature of ≤45° C., then mixing them to obtain a third solution, in which pH of the third solution is ≥7; and,
immersing the third solution in a deionized water solvent or an ethanol solvent to remove impurities to obtain a mineralized collagen gel.

Preferably, the method further comprises a step of freeze-drying the mineralized collagen gel to obtain a mineralized collagen scaffold.

Preferably, before the step of immersing the third solution in a deionized water solvent or an ethanol solvent, the method further comprises a step of allowing the third solution to stand at room temperature.

Preferably, a method of mixing the second solution and the first solution is selected from at least one of stirring, vortex oscillation and ultrasonic methods.

Preferably, a stirring time of mixing the second solution and the first solution is ≥1 min with a stirring speed of 10˜10,000 rpm.

Preferably, a dripping speed of adding the second solution to the first solution is 0.1˜10000 mL/min.

Preferably, in the first solution, the mass ratio of the glycerin to the first solvent is 50%˜100%; and/or,
the concentration of type I collagen is ≤100 mg/mL; and/or,
the concentration of calcium ion is ≤5 mol/L; and/or,
the type I collagen is selected from at least one of animal-derived collagen and recombinant collagen; and/or,
the calcium source is selected from at least one of calcium chloride, calcium bicarbonate, calcium bisulfate, calcium nitrate, calcium chlorate, calcium hypochlorite, calcium perchlorate, calcium bisulfite, calcium iodide, calcium bromide, and calcium permanganate; and/or,
the acid is selected from at least one of acetic acid, hydrochloric acid, nitric acid, and phosphoric acid.

Preferably, the type I collagen is selected from at least one of mouse-derived collagen, bovine-derived collagen and porcine-derived collagen.

Preferably, the concentration of the type I collagen is 1˜100 mg/mL.

Preferably, the concentration of the type I collagen is 5˜50 mg/mL.

Preferably, the concentration of the type I collagen is 10˜30 mg/mL.

Preferably, the concentration of the calcium ion is ≤3 mol/L.

Preferably, the concentration of the calcium ion is ≤2 mol/L.

Preferably, the first solution contains the acid with a concentration of 0.1M.

Preferably, in the second solution, the mass ratio of the glycerin to the second solvent is 0˜100%; and/or,
the concentration of phosphate ion is ≤3 mol/L; and/or,
the alkali is selected from at least one of sodium hydroxide, ammonia, and potassium hydroxide; and/or,
the phosphorus source is selected from at least one of phosphoric acid, sodium dihydrogen phosphate, trisodium phosphate, and sodium hydrogen phosphate.

Preferably, the concentration of the phosphate ion is ≤2 mol/L.

Preferably, the concentration of the phosphate ion is ≤1 mol/L.

Preferably, in the third solution, the molar ratio of calcium ion to phosphate ion is 0.1˜10:1; and/or,
the mass ratio of glycerin to water is ≥0.1:1.

Preferably, the molar ratio of calcium ion to phosphate ion is 0.3˜6:1.

Preferably, the molar ratio of calcium ion to phosphate ion is 0.5˜2:1.

Preferably, the mass ratio of glycerin to water is 0.1˜10:1.

Preferably, the mass ratio of glycerin to water is 0.2˜5:1.

Preferably, the mass ratio of glycerin to water is 0.4˜4:1.

Preferably, the mass ratio of glycerin to water is 0.5˜2:1.

Preferably, the second solution is added to the first solution at a temperature of 5˜30° C.

Preferably, the second solution is added to the first solution at a temperature of 10˜20° C.

A second aspect of the present invention provides a mineralized collagen material prepared by the method described in any one of the first aspect of the present invention.

A third aspect of the present invention provides a use of the mineralized collagen material in repairing bone defects, wherein the mineralized collagen material is prepared by the method described in any one of the first aspect of the present invention.

The present invention has the following advantages over the prior art: compared with related technologies, embodiments of this application provide a mineralized collagen material, preparation method and use thereof. In its preparation method, by introducing a certain amount of glycerin into a reaction system, an assembly speed and an assembly structure of the collagen can be controlled, nucleation and crystallization speeds and a structure of the mineral substance can be also controlled, so that the collagen can be assembled into a large-size space network structure at a controllable speed, and mineralization can be deeply performed simultaneously, so as to prepare continuous large-size mineralized collagen blocks. The product obtained in the present invention can be in a gel state with a self-supporting characteristics and a good flexibility. Meanwhile it can be formed into a preset three-dimensional shape through injection molding to meet different needs. And, it can also be in a porous bulk form (non-powder state) with an adjustable compressive strength and modulus after freeze-drying. In the absence of artificial chemical cross-linking, the porous structure can still be retained with a better flexibility after re-saturation and water absorption. The method is simple, efficient and controllable. By using glycerin, the formation of calcium phosphate can be controlled, and the collagen can be assembled into a large-sized, continuous spatial structure simultaneously, thereby forming a product that can be deeply and uniformly mineralized. Compared with ordinary water system mineralization, the method in the present invention can achieve controllable mineralization of collagen in a very small reaction system. The mineralization degree range is 1-80 wt %. Self-supporting mineralized collagen hydrogels or porous mineralized collagen bulk scaffolds can be produced without repeated replacement of a mineralizing solution, a macromolecular additive and an extra cross-linking.

### BRIEF DESCRIPTION OF THE DRAWINGS

The accompanying drawings described here are provided for further understanding of the present disclosure, and constitute a part of the present disclosure. The exemplary embodiments of the present disclosure and illustrations thereof are intended to explain the present disclosure, but do not constitute inappropriate limitations to the present disclosure. In the drawings:

FIG. 1a is a scanning electron microscope image of a mineralized collagen material according to one embodiment of the present invention.

FIG. 1b is a transmission electron microscope image of a mineralized collagen material according to one embodiment of the present invention.

FIG. 1c is an X-ray diffraction spectrum of a mineralized collagen material according to one embodiment of the present invention.

FIG. 2 is a scanning electron microscope image of a mineralized collagen material produced by a co-precipitation method in a conventional aqueous solution.

FIG. 3 is a schematic diagram of a mineralized collagen hydrogel obtained by a method for preparing a mineralized collagen material according to one embodiment of the present invention.

FIG. 4 is a schematic diagram of a mineralized collagen scaffold obtained by a method for preparing a mineralized collagen material according to one embodiment of the present invention.

FIG. 5 is a thermogravimetric analysis diagram of a mineralized collagen material according to one embodiment of the present invention.

### DETAILED DESCRIPTION

To make the objectives, technical solutions, and advantages of the present disclosure clearer, the present disclosure is described below with reference to the accompanying drawings and embodiments. It should be understood that the embodiments described herein are merely used to explain the present disclosure, rather than to limit the present disclosure. All other embodiments obtained by those of ordinary skill in the art based on the embodiments of the present disclosure without creative efforts should fall within the protection scope of the present disclosure.

Apparently, the accompanying drawings in the following description show merely some embodiments of the present disclosure, and a person of ordinary skill in the art may apply the present disclosure to other similar scenarios according to these drawings without creative efforts. In addition, it can also be appreciated that, although it may take enduring and complex efforts to achieve such a development process, for those of ordinary skill in the art related to the present disclosure, some changes such as design, manufacturing or production made based on the technical content in the present disclosure are merely regular technical means, and should not be construed as insufficiency of the present disclosure.

The “embodiment” mentioned in the present disclosure means that a specific feature, structure, or characteristic described in combination with the embodiment may be included in at least one embodiment of the present disclosure. The phrase appearing in different parts of the specification does not necessarily refer to the same embodiment or an independent or alternative embodiment exclusive of other embodiments. It may be explicitly or implicitly appreciated by those of ordinary skill in the art that the embodiment described herein may be combined with other embodiments as long as no conflict occurs.

Unless otherwise defined, the technical or scientific terms used in the present disclosure are as they are usually understood by those of ordinary skill in the art to which the present disclosure pertains. The terms “one”, “a”, “the” and similar words are not meant to be limiting, and may represent a singular form or a plural form. The terms “comprise”, “include”, “contain”, “have” and any other variants in the present disclosure mean to cover the non-exclusive inclusion, for example, a process, method, system, product, or device that includes a series of steps or modules (units) is not necessarily limited to those steps or units which are clearly listed, but may include other steps or units which are not expressly listed or inherent to such a process, method, system, product, or device. The term “and/or” describes associations between associated objects, and it indicates three types of relationships. For example, “A and/or B” may indicate that A exists alone, A and B coexist, or B exists alone. The character “/” generally indicates that the associated objects are in an “or” relationship. The terms “first”, “second”, “third” and so on in the present disclosure are intended to distinguish between similar objects but do not necessarily indicate a specific order of the objects.

### Embodiment 1

This embodiment is an illustrative example of the present invention and relates to a mineralized collagen material, preparation method and use thereof.

A method for preparing a mineralized collagen material comprises the steps of:
step S102: dissolving type I collagen, calcium source and acid in a first solvent to obtain a first solution, in which the first solvent is glycerin or a mixed solvent of glycerin and water;
step S104: dissolving alkali and phosphorus source in a second solvent to obtain a second solution, in which the second solvent is glycerin, water or a mixed solvent of glycerin and water;
step S106: adding the second solution to the first solution at a temperature of ≤45° C., then mixing them to obtain a third solution, in which pH of the third solution is ≥7; and,
step S108: immersing the third solution in a deionized water solvent or an ethanol solvent to remove impurities to obtain a mineralized collagen gel.

Wherein, in the first solution, the mass ratio of the glycerin to the first solvent is 50%˜100%; the concentration of the type I collagen is ≤100 mg/mL: the concentration of calcium ion is ≤5 mol/L: the type I collagen is selected from at least one of animal-derived collagen and recombinant collagen: the calcium source is selected from at least one of calcium chloride, calcium bicarbonate, calcium bisulfate, calcium nitrate, calcium chlorate, calcium hypochlorite, calcium perchlorate, calcium bisulfite, calcium iodide, calcium bromide, and calcium permanganate: the acid is selected from at least one of acetic acid, hydrochloric acid, nitric acid, and phosphoric acid; and, the first solution contains the acid with a concentration of 0.1M.

Preferably, the concentration of the type I collagen is 1˜100 mg/mL. More preferably, the concentration of the type I collagen is 5˜50 mg/mL. Most preferably, the concentration of type I collagen is 10˜30 mg/mL.

Preferably, the concentration of the calcium ion is ≤3 mol/L. More preferably, the concentration of the calcium ion is ≤2 mol/L.

Preferably, the type I collagen is selected from at least one of mouse-derived collagen, bovine-derived collagen and porcine-derived collagen.

Wherein, in the second solution, the mass ratio of the glycerin to the second solvent is 0˜100%: the concentration of phosphate ion is ≤3 mol/L: the alkali is selected from at least one of sodium hydroxide, ammonia, and potassium hydroxide: the phosphorus source is selected from at least one of phosphoric acid, sodium dihydrogen phosphate, trisodium phosphate, and sodium hydrogen phosphate.

Preferably, the concentration of the phosphate ion is ≤2 mol/L. More preferably, the concentration of phosphate ion is ≤1 mol/L

In some preferred embodiments, the concentration of the calcium ion is ≤5 mol/L, and the concentration of the phosphate ion is ≤3 mol/L.

In some preferred embodiments, the concentration of the calcium ion is ≤3 mol/L, and the concentration of the phosphate ion is ≤2 mol/L.

In some preferred embodiments, the concentration of the calcium ion is ≤2 mol/L, and the concentration of the phosphate ion is ≤1 mol/L.

Wherein, in the third solution, the molar ratio of calcium ion to phosphate ion is 0.1˜10:1; and, the mass ratio of glycerin to water is ≥0.1:1.

Preferably, the molar ratio of calcium ion to phosphate ion is 0.3˜6:1. More preferably, the molar ratio of calcium ion to phosphate ion is 0.5˜2:1.

Preferably, the mass ratio of glycerin to water is 0.1˜10:1. More preferably, the mass ratio of glycerin to water is 0.2˜5:1. Most preferably, the mass ratio of glycerin to water is 0.4˜4:1. Optimally, the mass ratio of glycerin to water is 0.5˜2:1.

Preferably, the second solution is added to the first solution at a temperature of 5˜30° C. More preferably, the second solution is added to the first solution at a temperature of 10˜20° C.

In some preferred embodiments, a method of mixing the second solution and the first solution comprises stirring method; and/or, vortex oscillation method; and/or, ultrasonic method.

In some preferred embodiments, in a stirring method, a stirring time of mixing the second solution and the first solution is ≥1 min with a stirring speed of 10˜10,000 rpm.

In some preferred embodiments, a dripping speed of adding the second solution to the first solution is 0.1˜10000 mL/min.

In step S106, the third solution is basically in a gel form.

In step S108, the purpose of placing the third solution in the deionized water solvent or ethanol solvent is to remove impurities, including glycerin, alkali, salts generated by the reaction of alkali and acid (basically inorganic salts), unreacted calcium source and unreacted phosphorus source.

In step S108, the third solution is placed in the deionized water solvent or ethanol solvent for repeated immersion.

Specifically, after immersing the third solution in the deionized water solvent or ethanol solvent for a certain period of time, the third solution is taken out and immersed again in a new deionized water solvent or ethanol solvent, repeated for many times.

In some preferred embodiments, the ethanol solvent can also be used for gradient immersing of the third solvent, that is, the concentration/mass ratio of the ethanol solvent for each immersion is progressively increased, for example, the ethanol solvent for the first immersing is 75% ethanol, and the ethanol solvent for the second immersing is 80% ethanol, and the ethanol solvent for the last immersing is 100% ethanol.

Preferably, the method further comprises:
step S107: allowing the third solution to stand at room temperature.

The purpose of the standing is to make the third solution be in a gel form.

Preferably, the time for standing at room temperature is ≥8 h. More preferably, the time for standing at room temperature is ≥24 h.

Preferably, the method further comprises:
step S110: freeze-drying the mineralized collagen gel to obtain a mineralized collagen scaffold.

In step S110, the obtained mineralized collagen scaffold is a porous structure with an increased specific surface area, which makes it easier to repair bone defects.

In some preferred embodiments, the degree of mineralization of the mineralized collagen gel/mineralized collagen scaffold is >1%.

Preferably, the degree of mineralization of the mineralized collagen gel/mineralized collagen scaffold is >40%.

More preferably, the degree of mineralization of the mineralized collagen gel/mineralized collagen scaffold is >65%.

The obtained mineralized collagen material includes the mineralized collagen gel and the mineralized collagen scaffold.

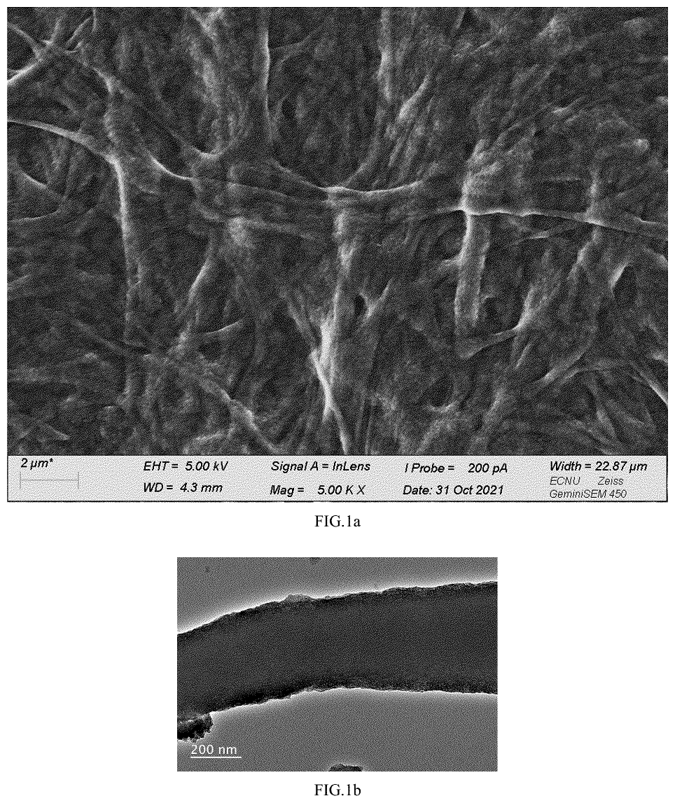

Specifically, as shown in FIG. 1a, the scanning electron microscope image of a mineralized collagen material produced by the present method shows that all collagen fibers are uniformly and completely wrapped by calcium phosphate. As shown in FIG. 1b, the transmission electron microscope image of the mineralized collagen material shows that the entire collagen fibers have been completely mineralized, and the calcium phosphate components within the fiber bundles are evenly distributed. As shown in FIG. 1c, the XRD of the mineralized collagen material shows a typical hydroxyapatite crystallization peak, confirming that the mineralized substance is hydroxyapatite. The combination of the above three characterization methods shows that the mineralized collagen material has the characteristics of high mineralization, uniformity and completeness.

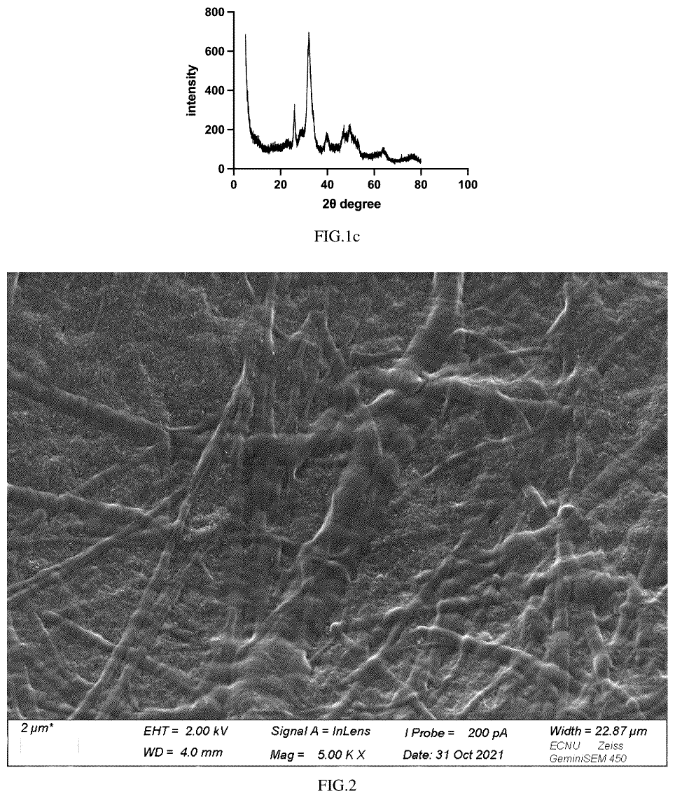

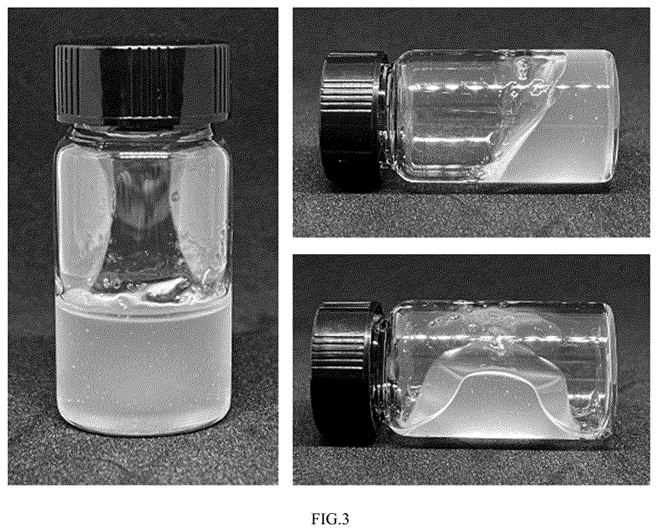

As shown in FIG. 2, the scanning electron microscope image of the mineralized collagen material prepared by the traditional co-precipitation method shows exposed collagen fibers and their characteristic strips, proving that the mineralization is uneven and incomplete. Compared with the mineralized collagen material prepared by the present invention, it shows that the mineralized collagen material prepared by the present invention has a high degree of mineralization, and is uniform and complete.

As shown in FIG. 3, the mineralized collagen hydrogel produced by the present method has a certain mechanical strength without cross-linking, which can maintain its own integrity.

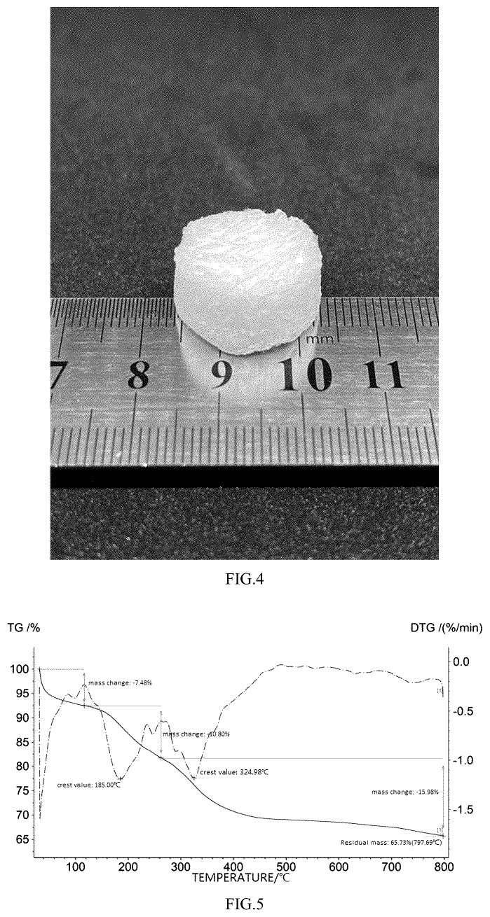

As shown in FIG. 4, the mineralized collagen scaffold made by the present method has characteristics of uniformity and completeness.

As shown in FIG. 5, the thermogravimetric analysis shows that the mineralization degree of the present method is relatively high, even higher than 65%.

For the above-mentioned mineralized collagen material, it can be applied to repair bone defects.

Specifically, the mineralized collagen material can be used as a single component or in combination with other components.

The present invention provides a method for preparing mineralized collagen that is simple, efficient and suitable for industrial mass production. In the preparation method, by introducing a certain amount of glycerin into a reaction system, an assembly speed and an assembly structure of the collagen can be controlled, nucleation and crystallization speeds and a structure of the mineral substance can be also controlled, so that the collagen can be assembled into a large-size space network structure at a controllable speed, and mineralization can be deeply performed simultaneously, so as to prepare continuous large-size mineralized collagen blocks. The product obtained in the present invention can be in a gel state with a self-supporting characteristics and a good flexibility. Meanwhile, it can be formed into a preset three-dimensional shape through injection molding to meet different needs. And, it can also be in a porous bulk form (non-powder state) with an adjustable compressive strength and modulus after freeze-drying. In the absence of artificial chemical cross-linking, the porous structure can still be retained with a better flexibility after re-saturation and water absorption. The method is simple, efficient and controllable. By using glycerin, the formation of calcium phosphate can be controlled, and the collagen can be assembled into a large-sized, continuous spatial structure simultaneously, thereby forming a product that can be deeply and uniformly mineralized. Compared with ordinary water system mineralization, the method in the present invention can achieve controllable mineralization of collagen in a very small reaction system. The content of the mineralization component is 1-80 wt %. Self-supporting mineralized collagen hydrogels or porous mineralized collagen bulk scaffolds can be produced without repeated replacement of a mineralizing solution, a macromolecular additive and an extra cross-linking.

### Embodiment 2

This embodiment is a specific example of the present invention.

At room temperature and under stirring with a magnetic stirring rod (500-600 rpm), calcium chloride, porcine-derived collagen and acetic acid are added to a glycerin/water mixture (the mass ratio of glycerin to water is 2:3) to obtain a first solution containing 0.06M calcium chloride, 0.1M acetic acid and 15 mg/mL collagen.

Trisodium phosphate and sodium hydroxide are dissolved in glycerin to obtain a second solution containing 0.05M trisodium phosphate and 0.5M sodium hydroxide.

At 10° C., under stirring with a magnetic stirring rod (800-1000 rpm), 3.2 mL of the second solution is slowly dropped into 4.6 mL of the first solution, mixing them thoroughly to obtain a third solution.

The third solution is allowed to stand at room temperature. After 1 day, the content is taken out and immersed repeatedly in deionized water, and then freeze-dried to prepare a mineralized collagen scaffold.

### Embodiment 3

This embodiment is a specific example of the present invention.

At room temperature and under stirring with a magnetic stirring rod (500-600 rpm), calcium chloride, mouse-derived collagen and acetic acid are added to deionized water to obtain a first solution containing 0.1M calcium chloride, 0.1M acetic acid and 5 mg/mL collagen.

Trisodium phosphate and sodium hydroxide are dissolved in glycerin to obtain a second solution containing 0.05M trisodium phosphate and 0.5M sodium hydroxide.

At 10° C., under stirring with a magnetic stirring rod (800-1000 rpm), 3.2 mL of the second solution is slowly dropped into 5.0 mL of the first solution, mixing them thoroughly to obtain a third solution.

The third solution is allowed to stand at room temperature. After 2 days, the content is taken out and immersed repeatedly in deionized water, and then freeze-dried to prepare a mineralized collagen scaffold.

### Embodiment 4

This embodiment is a specific example of the present invention.

At room temperature and under stirring with a magnetic stirring rod (500-600 rpm), calcium chloride, porcine-derived collagen and acetic acid are added to a glycerin/water mixture (the mass ratio of glycerin to water is 1:3) to obtain a first solution containing 2M calcium chloride, 0.1M acetic acid and 20 mg/mL collagen.

Trisodium phosphate and sodium hydroxide are dissolved in glycerin to obtain a second solution containing 1M trisodium phosphate and 1M sodium hydroxide.

At 10° C., under stirring with a magnetic stirring rod (800-1000 rpm), 3.2 mL of the second solution is slowly dropped into 4.6 mL of the first solution, mixing them thoroughly to obtain a third solution.

The third solution is allowed to stand at room temperature. After 5 days, the content is taken out and immersed repeatedly in deionized water, and then freeze-dried to prepare a mineralized collagen scaffold.

### Embodiment 5

This embodiment is a specific example of the present invention.

At room temperature and under stirring with a magnetic stirring rod (500-600 rpm), calcium nitrate, bovine-derived collagen and acetic acid are added to the glycerin to obtain a first solution containing 1M calcium nitrate, 0.1M acetic acid and 5 mg/mL collagen.

Trisodium phosphate and sodium hydroxide are dissolved in deionized water to obtain a second solution containing 0.5M trisodium phosphate and 0.2M sodium hydroxide.

At 10° C., under stirring with a magnetic stirring rod (800-1000 rpm), 4.9 mL of the second solution is slowly dropped into 3.9 mL of the first solution, mixing them thoroughly to obtain a third solution.

The third solution is allowed to stand at room temperature. After 3 days, the content is taken out and immersed repeatedly in deionized water, and then freeze-dried to prepare a mineralized collagen scaffold.

### Embodiment 6

This embodiment is a specific example of the present invention.

At room temperature and under stirring with a magnetic stirring rod (500-600 rpm), calcium permanganate, mouse-derived collagen and hydrochloric acid are added to a glycerin/water mixture (the mass ratio of glycerin to water is 2:3) to obtain a first solution containing 1M calcium permanganate, 0.1M hydrochloric acid and 15 mg/mL collagen.

Sodium hydrogen phosphate and sodium hydroxide are dissolved in deionized water to obtain a second solution containing 0.5M sodium hydrogen phosphate and 0.3M sodium hydroxide.

At 10° C., under stirring with a magnetic stirring rod (800-1000 rpm), 5.6 mL of the second solution is slowly dropped into 1.8 mL of the first solution, mixing them thoroughly to obtain a third solution.

The third solution is allowed to stand at room temperature. After 0.5 day, the content is taken out and immersed repeatedly in deionized water, and then freeze-dried to prepare a mineralized collagen scaffold.

### Embodiment 7

This embodiment is a specific example of the present invention.

At room temperature and under stirring with a magnetic stirring rod (500-600 rpm), calcium chloride, mouse-derived collagen and acetic acid are added to the glycerin to contain a first solution containing 0.3M calcium chloride, 0.1M acetic acid and 10 mg/mL collagen.

Trisodium phosphate and sodium hydroxide are dissolved in deionized water to obtain a second solution containing 3M trisodium phosphate and 0.5M sodium hydroxide.

At 10° C., under stirring with a magnetic stirring rod (800-1000 rpm), 1.0 mL of the second solution is slowly dropped into 8.66 mL of the first solution, mixing them thoroughly to obtain a third solution.

The third solution is allowed to stand at room temperature. After 1 day, the content is taken out and immersed repeatedly in deionized water, and then freeze-dried to prepare a mineralized collagen scaffold.

### Embodiment 8

This embodiment is a specific example of the present invention.

At room temperature and under stirring with a magnetic stirring rod (500-600 rpm), calcium chloride, mouse-derived collagen and acetic acid are added to a glycerin/water mixture (the mass ratio of glycerin to water is 1:1) to obtain a first solution containing 1M calcium chloride, 0.1M acetic acid and 20 mg/mL collagen.

Trisodium phosphate and sodium hydroxide are dissolved in glycerin to obtain a second solution containing 0.1M trisodium phosphate and 0.5M sodium hydroxide.

At 10° C., under stirring with a magnetic stirring rod (800-1000 rpm), 3.2 mL of the second solution is slowly dropped into 3.7 mL of the first solution, mixing them thoroughly to obtain a third solution.

The third solution is allowed to stand at room temperature. After 1 day, the content is taken out and immersed repeatedly in deionized water, and then freeze-dried to prepare a mineralized collagen scaffold.

The above embodiments are merely illustrative of several implementation manners of the present disclosure, and the description thereof is more specific and detailed, but is not to be construed as a limitation to the patentable scope of the present disclosure. It should be pointed out that several variations and improvements can be made by those of ordinary skill in the art without departing from the conception of the present disclosure, but such variations and improvements should fall within the protection scope of the present disclosure. Therefore, the protection scope of the patent of the present disclosure should be subject to the appended claims.

1. A method for preparing a mineralized collagen material, wherein comprising the steps of:
dissolving type I collagen, calcium source and acid in a first solvent to obtain a first solution, in which the first solvent is glycerin or a mixed solvent of glycerin and water;
dissolving alkali and phosphorus source in a second solvent to obtain a second solution, in which the second solvent is glycerin, water or a mixed solvent of glycerin and water;
adding the second solution to the first solution at a temperature of ≤45° C., then mixing them to obtain a third solution, in which pH of the third solution is ≥7; and,
immersing the third solution in a deionized water solvent or an ethanol solvent to remove impurities to obtain a mineralized collagen gel.

2. he method for preparing a mineralized collagen material according to claim 1, wherein the method further comprising a step of:
freeze-drying the mineralized collagen gel to obtain a mineralized collagen scaffold.

3. The method for preparing a mineralized collagen material according to claim 1, wherein the method further comprising a step of:
allowing the third solution to stand at room temperature before the step of immersing the third solution in a deionized water solvent or an ethanol solvent.

4. The method for preparing a mineralized collagen material according to claim 1, wherein a method of mixing the second solution and the first solution is selected from at least one of stirring, vortex oscillation and ultrasonic methods.

5. The method for preparing a mineralized collagen material according to claim 1, wherein a dripping speed of adding the second solution to the first solution is 0.1˜10000 mL/min.

6. The method for preparing a mineralized collagen material according to claim 1, wherein in the first solution,
the mass ratio of the glycerin to the first solvent is 50%˜100%; and/or, the concentration of the type I collagen is ≤100 mg/mL; and/or,
the concentration of calcium ion is ≤5 mol/L; and/or,
the type I collagen is selected from at least one of animal-derived collagen and recombinant collagen; and/or,
the calcium source is selected from at least one of calcium chloride, calcium bicarbonate, calcium bisulfate, calcium nitrate, calcium chlorate, calcium hypochlorite, calcium perchlorate, calcium bisulfite, calcium iodide, calcium bromide, and calcium permanganate; and/or,
the acid is selected from at least one of acetic acid, hydrochloric acid, nitric acid, and phosphoric acid.

7. The method for preparing a mineralized collagen material according to claim 1, wherein in the second solution, the mass ratio of the glycerin to the second solvent is 0˜100%; and/or,
the concentration of phosphate ion is ≤3 mol/L; and/or,
the alkali is selected from at least one of sodium hydroxide, ammonia, and potassium hydroxide; and/or,
the phosphorus source is selected from at least one of phosphoric acid, sodium dihydrogen phosphate, trisodium phosphate, and sodium hydrogen phosphate.

8. The method for preparing a mineralized collagen material according to claim 1, wherein in the third solution, the molar ratio of calcium ion to phosphate ion is 0.1˜10:1; and/or,
the mass ratio of glycerin to water is ≥0.1:1.

9. A mineralized collagen material prepared by the method described in claim 1.

10. Use of a mineralized collagen material in repairing bone defects, wherein the mineralized collagen material is prepared by the method according to claim 1, and the use comprises a step of administering an effective dose of the mineralized collagen material to a patient.
